(19) United States
(12) Patent Application Publication
(10) Pub. No.: US 2019/0033315 A1
(43) Pub. Date: Jan. 31, 2019

(54) METABOLOMICS FOR DIAGNOSING PANCREATIC CANCER

(71) Applicant: UTI LIMITED PARTNERSHIP, Calgary (CA)

(72) Inventors: Oliver F. BATHE, Calgary (CA); Yarrow MCCONNELL, Calgary (CA); Rustem SHAYKHUTDINOV, Calgary (CA); Karen KOPCIUK, Calgary (CA); Aalim M. WELJIE, Calgary (CA); Hans J. VOGEL, Calgary (CA)

(73) Assignee: UTI LIMITED PARTNERSHIP, Calgary, AB (CA)

(21) Appl. No.: 15/535,222

(22) PCT Filed: Dec. 16, 2015
(86) PCT No.: PCT/IB2015/002486

§ 371 Date: Jan. 24, 2018

Related U.S. Application Data
(60) Provisional application No. 62/094,700, filed on Dec. 19, 2014.

(51) Int. Cl. G01N 33/574; G01N 33/483; G01N 33/92; G01N 33/68; G01N 30/02; G01N 33/50

(52) U.S. Cl. CPC G01N 33/57438; G01N 33/483; G01N 33/92; G01N 33/6842; G01N 30/02; G01N 33/507; G01N 2800/60; G01N 33/6848

(57) ABSTRACT

The present disclosure is drawn to methods of diagnosing and classifying pancreatic cancer by examining the expression of particular metabolites that distinguish this disease state from benign disease and periampullary adenocarcinoma.

This application is a national phase application under 35 U.S.C. § 371 of International Application No. PCT/IB2015/002486, filed Dec. 16, 2015, which claims benefit of priority to U.S. Provisional Application Serial No. 62/094,700, filed Dec. 19, 2014, the entire contents of each are hereby incorporated by reference.

### BACKGROUND

### I. Field

The present disclosure relates generally to the fields of biochemistry, molecular biology, and medicine. In certain aspects, the disclosure is related to to use of a panel of metabolites whose expression is diagnostic for pancreatic cancer and cancer types.

### II. Description of Related Art

Patients with lesions of the pancreas or periampullary structures may present with jaundice and/or pain, or lesions can be found incidentally on imaging. Periampullary lesions may arise from the distal common bile duct, ampulla of Vater, or the duodenum. In each case, the major diagnostic consideration is to distinguish between malignant lesions (especially adenocarcinoma) and benign lesions. Malignant lesions warrant early surgical consideration. Benign lesions, such as pancreatitis, benign strictures, and serous cysts, are typically treated non-operatively.

Current diagnostic approaches, variously combining clinical examination, cross-sectional imaging, endoscopic retrograde cholangiopancreatography (ERCP) with brush biopsy, endoscopic ultrasound (EUS) with fine needle aspiration (FNA), and serum CA 19-9 can accurately distinguish benign from malignant lesions 60-90% of the time (Goonetilleke and Siriwardena, 2007; Kinney, 2010; Savides et al., 2007; Fogel et al., 2006). Such extensive diagnostic investigations can delay definitive surgery for patients who are ultimately proven to have a pancreatic or periampullary cancer, potentially affecting their outcome. In addition, when the diagnosis remains unclear despite extensive investigation, most patients and surgeons opt for surgical exploration with possible resection. This results in a finding of benign pathology in 7-13% of pancreatic surgical resection specimens (Abraham et al., 2003; Yeo et al., 1997; Camp and Vogel, 2004). Given that pancreatic surgery is associated with substantial morbidity and a significant risk of perioperative mortality (Simunovic et al., 2010; Simons et al., 2009), a reduction in the need for such “diagnostic” resections would be beneficial. Therefore, better non-invasive diagnostic tests that accurately differentiate malignant from benign pancreatic lesions are clearly needed.

### SUMMARY

Thus, in accordance with the present disclosure, there is provided a method of distinguishing pancreatic cancer and periampullary adenocarcinoma from benign pancreatic lesions in a subject comprising (a) obtaining a blood, plasma or serum sample; (b) determining the levels of 4 or more of the biomarkers set forth in Table 3 in said sample; and (c) assigning to said sample a classification as (i) pancreatic cancer or periampullary adenocarcinoma where one or more markers indicating pancreatic cancer or periampullary adenocarcinoma are elevated; and (ii) benign pancreatic lesion where one or more markers indicating benign pancreatic lesions are elevated. In particular, step (b) may comprise determining the levels of Galactose, Unmatched RI:1007.82 QI: 67/82/83, Isopropanol, Mannose, Trimethylamine-N-oxide, Arabitol, Threitol, Succinate, Trehalose-alpha, Match RI:2018.25 QI: 191/217/305/318/507, Tridecanol, Azelaic acid, Unmatched RI:2475.33 QI: 73/375/376, Pyroglutamate, Isoleucine, Tyrosine, Arginine, Unmatched RI:1913.88 QI: 156/174/317, Alanine, Creatine, Lysine, Unmatched RI:1971.99 QI: 185/247/275; and step (c) comprises assigning to said sample a classification as pancreatic cancer or periampullary adenocarcinoma where a pluraliity of Galactose, Unmatched RI:1007.82 QI: 67/82/83, Isopropanol, Mannose, Trimethylamine-N-oxide, Arabitol, Threitol, Succinate, and Trehalose-alpha levels are elevated; and benign pancreatic lesion where a plurality of Match RI:2018.25 QI: 191/217/305/318/507, Tridecanol, Azelaic acid, Unmatched RI:2475.33 QI: 73/375/376, Pyroglutamate, Isoleucine, Tyrosine, Arginine, Unmatched RI:1913.88 QI: 156/174/317, Alanine, Creatine, Lysine, Unmatched RI:1971.99 QI: 185/247/275 levels are elevated. The information may be determined using gas chromatography/mass spectrometry or nuclear magnetic resonance or both. Information on the level of at least 10, 14, 15, 18, 20, 25 or all 30 of the metabolites from Table 3 may be determined.

The levels may be compared to a pre-determined standard, or from a serum, plasma or blood sample from a non-cancer subject. The method may further comprise obtaining said serum, plasma or blood sample from said subject. The pancreatic cancer may be metastatic or localized. The method may further comprise treating said subject for pancreatic cancer, such as with surgery, chemotherapy or both. The non-pancreatic cancer may be a benign pancreatic lesion, or a periampullary adenocarcinoma, such as ampullary cancer, bile duct cancer or duodenal cancer.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIGS. 1A-B. Principal components analysis (PCA) results. Scatter plots showing scores (t) in first two components of PCA models for one training dataset (FIG. 1A: ¹H-NMR; FIG. 1B: GC-MS). Results from other training sets were similar. Plots coded for patient diagnosis: malignant: ▴ vs. benign: ⋄.

FIGS. 2A-C. Orthogonal partial least squares discriminant analysis (OPLS-DA) result. Scatter plots showing scores (t) in first (t[1]) and orthogonal (to[1]) components of final OPLS-DA models for one training dataset (a: ¹H-NMR, b: GC-MS, c: Combined). Results from other training sets were similar. Plots coded for patient diagnosis: malignant: ▴ vs. benign: ⋄.

FIG. 3. Whisker box limits are 1st and 3rd quartiles, whisker bar limits are upper and lower adjacent values. Middle line is median. Y-axis displays normalized concentration/ion abundance data: NMR=μmol, GC-MS=ion abundance. Benign disease: light gray, malignant disease: dark gray

### DESCRIPTION OF ILLUSTRATIVE EMBODIMENTS

Multi-marker panels are providing improved diagnostic accuracy in several medical fields. Parallel to the development of genomic and proteomic multi-marker panels, metabolomic approaches are now being used to identify panels of low molecular weight compounds that characterize particular disease states. “Metabolomics” describes the “quantitative measurement of time-related multiparametric metabolic responses of multicellular systems to pathophysiological stimuli or genetic modification” (Nicholson et al., 1999). The biomarkers of interest consist of metabolites, small molecules which are intermediates and products of metabolism, including molecules associated with energy storage and utilization; precursors to proteins and carbohydrates; regulators of gene expression; and signaling molecules. Thus, like the proteome, the metabolome represents a functional portrait of the cell or the organism. Changes in metabolism result in alterations of the abundance of groups of metabolites. Therefore, patterns of changes in metabolites associated with a particular disease state could provide a biomarker of considerable interest.

Metabolomic techniques have demonstrated the ability to distinguish a number of disease processes, including breast and prostate cancer (Dunn et al., 2007; Fiehn et al., 2010; Xue et al., 2009; Asiago et al., 2010; Streekumar et al., 2009). In patients with colorectal cancer, compared to normal controls, metabolomics techiques could distinguish serum metabolite differences with 75% sensitivity and 90% specificity (Ritchie et al., 2010). The finding of a similarly accurate serum metabolite profile to differentiate pancreatic cancer from other pancreatic lesions could lead to the development of a highly useful diagnostic tool.

A metabolomic biomarker is very different from a proteomic biomarker and a transcriptomic biomarker; its features will enhance its power as a biomarker. A metabolomic biomarker is not just a string of changes in individual metabolites. Rather, it is comprised of groups of co-related metabolites that change in concert; it is a meta-biomarker. For example, changes in circulating metabolites associated with CRC might reflect alterations in metabolism that are contained within tumor as well as alterations in the general health of the host, producing an overall “tumor signal” that reflects the extent of disease as well as its biology. In a person receiving chemotherapy, several discreet processes can be followed at once, including appearance of cell death, reduction in cell proliferation, and reduction in “tumor signal.” Importantly, because a metabolomic biomarker is a meta-biomarker, a random change in a single metabolite will not provide a false signal. A metabolomic biomarker therefore represents a powerful means of monitoring changes in an individual's condition over time.

The inventors considered that a metabolomic approach would be useful in the investigation of pancreatic cancer for a number of reasons. Pancreatic cancer is well known to have associated metabolic changes. The prevalence of diabetes mellitus in pancreatic cancer is reported as 40-47%, often preceding the diagnosis within less than 2 years (Pannala et al., 2008; Chari et al., 2008; Chari et al., 2005). Hyperinsulinemia and peripheral insulin resistance are typical in pancreatic cancer, whereas chronic pancreatitis (which also may be associated with a pancreatic mass) is accompanied by islet cell destruction and impaired insulin production (Meisterfeld et al., 2008; Larsen, 1993). Serum lactate levels tend to be higher in patients with periampullary malignancies compared to healthy controls and patients with benign periampullary lesions (Nishijima et al., 1997). In animal models of pancreatic cancer (Fang et al., 2007), metabolomic profiles associated with disease progression have been demonstrated. Others have reported that the metabolomic profile of bile can discriminate benign and malignant strictures (Fang et al., 2007; Wen et al., 2009). However, bile is generally inconvenient to sample. Recently, it was demonstrated in a small number of patients with pancreatic cancer that the salivary metabolomic profile was significantly different from normal controls (Sugimoto et al., 2010). Finally, the plasma metabolomic profiles of 5 patients (incidence of diabetes and jaundice unknown) were significantly different from normal controls (Urayama et al., 2010). These early results spurred the inventors' interest in more fully exploring the feasibility of using serum metabolomics to improve the diagnosis of pancreatic cancer.

In metabolomics studies using serum, samples are most frequently submitted to nuclear magnetic resonance spectroscopy (NMR) and/or gas chromotography-mass spectrometry (GC-MS) for metabolite detection. The resulting data sets contain information on hundreds of metabolites at wide-ranging concentrations. Complex data processing and statistical analysis is required to determine which metabolites differentiate patients with the disease, from those without.(Wishart, 2010) NMR is a widely-used technique due to its reliability and its ability to measure metabolite concentrations. There are established methods for quantitative analysis of NMR metabolomics datasets (Weljie et al., 2006; Trygg et al., 2007). GC-MS is a well recognized technique for metabolite detection although methods for untargeted GC-MS analyses are less well established. Although GC-MS is not a directly quantitative technique, it is much more sensitive than NMR and detects different subgroups of metabolites (Trygg et al., 2005; Begley et al., 2009). The inventors therefore used both NMR and GC-MS, as complimentary techniques.

The inventors' previous work has demonstrated the ability of proton nuclear magnetic resonance (¹H-NMR) spectroscopy to differentiate serum samples from patients with pancreatic/periampullary cancer versus benign pancreatic/hepatobiliary disease using 22 metabolites and achieving an internal AUROC of 0.83.¹⁰ The current study is an extension of that work. It aims to further investigate and refine the specific metabolomic profile of malignant versus benign pancreatic/periampullary lesions by excluding patients with hepatic or proximal biliary disease, and including an additional 90 patients with malignant or benign pancreatic or periampullary lesions.

Serum samples from this larger cohort of patients with exclusively pancreatic or periampullary lesions were interrogated using both ¹H-NMR spectroscopy and gas chromatography-mass spectrometry (GC-MS). GC-MS has the potential to strengthen the final metabolomic profile due to its greater sensitivity and ability to detect different metabolites than ¹H-NMR spectroscopy.^{11 1}H-NMR spectroscopy and GC-MS results were analyzed separately as well as in a combined fashion, to evaluate their relative strength and potential synergism.

Using multivariate projection modeling techniques, the minimal list of metabolites that consistently distinguished serum from patients with malignant versus benign pancreatic/periampullary lesions was developed in randomly allocated training sets. Separate test sets of patients were used to validate the resulting profile, and pathway analysis was performed to explore altered metabolic functions in malignant versus benign samples.

These and other aspects of the disclosure are described in detail below.

### I. PANCREATIC CANCER

Pancreatic cancer refers to a malignant neoplasm of the pancreas. The most common type of pancreatic cancer, accounting for 95% of these tumors is adenocarcinoma, which arises within the exocrine component of the pancreas. A minority arises from the islet cells and is classified as a neuroendocrine tumor. The symptoms that lead to diagnosis depend on the location, the size, and the tissue type of the tumor. They may include abdominal pain and jaundice (if the tumor compresses the bile duct).

Pancreatic cancer is the fourth most common cause of cancer death across the globe. Pancreatic cancer often has a poor prognosis: for all stages combined, the 1- and 5-year relative survival rates are 25% and 6%, respectively; for local disease the 5-year survival is approximately 20% while the median survival for locally advanced and for metastatic disease, which collectively represent over 80% of individuals, is about 10 and 6 months respectively. In 2010, an estimated 43,000 people in the U.S. were diagnosed with pancreas cancer and almost 37,000 died from the disease; pancreatic cancer has one of the highest fatality rates of all cancers, and is the fourth-highest cancer killer among both men and women worldwide. Although it accounts for only 2.5% of new cases, pancreatic cancer is responsible for 6% of cancer deaths each year.

Pancreatic cancer is sometimes referred to as a “silent killer” because early pancreatic cancer often does not cause symptoms, and the later symptoms are usually nonspecific and varied. Therefore, pancreatic cancer is often not diagnosed until it is advanced. Common symptoms include pain in the upper abdomen that typically radiates to the back (seen in carcinoma of the body or tail of the pancreas), loss of appetite and/or nausea and vomiting, significant weight loss, and painless jaundice (yellow tint to whites of eyes and/or yellowish skin in serious cases, possibly in combination with darkened urine) when a cancer of the head of the pancreas (75% of cases) obstructs the common bile duct as it runs through the pancreas. This may also cause pale-colored stool and steatorrhea. The jaundice may be associated with itching as the salt from excess bile can cause skin irritation.

Trousseau sign, in which blood clots form spontaneously in the portal blood vessels, the deep veins of the extremities, or the superficial veins anywhere on the body, is sometimes associated with pancreatic cancer. Diabetes mellitus or elevated blood sugar level are other possible indicators. Many patients with pancreatic cancer develop diabetes months to even years before they are diagnosed with pancreatic cancer, suggesting new onset diabetes in an elderly individual may be an early warning sign of pancreatic cancer. Clinical depression has been reported in association with pancreatic cancer, sometimes presenting before the cancer is diagnosed. However, the mechanism for this association is not known.

Risk factors for pancreatic cancer may include:
Family history: 5-10% of pancreatic cancer patients have a family history of pancreatic cancer. The genes have not been identified. Pancreatic cancer has been associated with the following syndromes: autosomal recessive ataxia-telangiectasia and autosomal dominantly inherited mutations in the BRCA2 gene and PALB2 gene, Peutz-Jeghers syndrome due to mutations in the STK11 tumor suppressor gene, hereditary non-polyposis colon cancer (Lynch syndrome), familial adenomatous polyposis, and the familial atypical multiple mole melanoma-pancreatic cancer syndrome (FAMMM-PC) due to mutations in the CDKN2A tumor suppressor gene. There may also be a history of familial pancreatitis.
Age: The risk of developing pancreatic cancer increases with age. Most cases occur after age 60, while cases before age 40 are uncommon.
Smoking: Cigarette smoking has a risk ratio of 1.74 with regard to pancreatic cancer; a decade of nonsmoking after heavy smoking is associated with a risk ratio of 1.2.
Diet: diets low in vegetables and fruits, high in red meat, and high in sugar-sweetened drinks (soft drinks)—risk ratio 1.87. In particular, the common soft drink sweetener fructose has been linked to growth of pancreatic cancer cells.
Obesity
Diabetes: Diabetes mellitus is both risk factor for pancreatic cancer, and, as noted earlier, new onset diabetes can be an early sign of the disease.
Helicobacter pylori infection
Gingivitis or periodontal disease

Most patients with pancreatic cancer experience pain, weight loss, or jaundice. Chronic pancreatitis has been linked, but is not known to be causal. The risk of pancreatic cancer in individuals with familial pancreatitis is particularly high.

Pain is present in 80% to 85% of patients with locally advanced or advanced metastatic disease. The pain is usually felt in the upper abdomen as a dull ache that radiates straight through to the back. It may be intermittent and made worse by eating. Weight loss can be profound; it can be associated with anorexia, early satiety, diarrhea, or steatorrhea. Jaundice is often accompanied by pruritus and dark urine. Painful jaundice is present in approximately one-half of patients with locally unresectable disease, while painless jaundice is present in approximately one-half of patients with a potentially resectable and curable lesion.

The initial presentation varies according to location of the cancer. Malignancies in the pancreatic body or tail usually present with pain and weight loss, while those in the head of the gland typically present with steatorrhea, weight loss, and jaundice. The recent onset of atypical diabetes mellitus, a history of recent but unexplained thrombophlebitis (Trousseau sign), or a previous attack of pancreatitis are sometimes noted. Courvoisier sign defines the presence of jaundice and a painlessly distended gallbladder as strongly indicative of pancreatic cancer, and may be used to distinguish pancreatic cancer from gallstones. Tiredness, irritability and difficulty eating because of pain also exist. Pancreatic cancer is often discovered during the course of the evaluation of aforementioned symptoms.

Liver function tests can show a combination of results indicative of bile duct obstruction (raised conjugated bilirubin, γ-glutamyl transpeptidase and alkaline phosphatase levels). CA19-9 (carbohydrate antigen 19.9) is a tumor marker that is frequently elevated in pancreatic cancer. However, it lacks sensitivity and specificity. When a cutoff above 37 U/mL is used, this marker has a sensitivity of 77% and specificity of 87% in discerning benign from malignant disease. CA 19-9 might be normal early in the course, and could be elevated because of benign causes of biliary obstruction. [Imaging studies, such as computed tomography (CT scan) and endoscopic ultrasound (EUS) can be used to identify the location and form of the cancer.

The most common form of pancreatic cancer (ductal adenocarcinoma) is typically characterized by moderately to poorly-differentiated glandular structures on microscopic examination. Pancreatic cancer has an immunohistochemical profile that is similar to hepatobiliary cancers (e.g., cholangiocarcinoma) and some stomach cancers; thus, it may not always be possible to be certain that a tumour found in the pancreas arose from it.

Pancreatic carcinoma is thought to arise from progressive tissue changes. Three types of precancerous lesion are recognised: pancreatic intraepithelial neoplasia—a microscopic lesion of the pancreas, intraductal papillary mucinous neoplasms and mucinous cystic neoplasms both of which are macroscopic lesions. The cellular origin of these lesions is debated.

The second most common type of exocrine pancreas cancer is mucinous. The prognosis is slightly better. Other exocrine cancers include adenosquamous carcinomas, signet ring cell carcinomas, hepatoid carcinomas, colloid carcinomas, undifferentiated carcinomas, and undifferentiated carcinomas with osteoclast-like giant cells.

Pancreatic endocrine tumors (PETs) are also called pancreatic neuroendocrine tumors (PNETs) and islet cell tumors. The annual clinically recognized incidence is low, about five per one million person-years. However, autopsy studies incidentally identify PETs in up to 1.5% most of which would remain inert and asymptomatic.

The more aggressive endocrine pancreatic cancers are known as pancreatic neuroendocrine carcinomas (PNEC). Similarly, there has likely been a degree of admixture of PNEC and extrapulmonary small cell cancer.

Diagnostic challenges. When a clinician encounters a pancreatic mass or a biliary stricture, it is often very difficult to determine whether the lesion is pancreatic adenocarcinoma, which requires timely and specific intervention. Obtaining a reliable tissue diagnosis is extremely difficult. Bile duct brushings only have a yield of 23-41% (Fogel et al., 2006; Mahmoudi et al., 2008). The diagnostic rate of biopsies for pancreatic masses is only about 71% (Savides et al., 2007). Moreover, while the sensitivity of biopsies is about 85%, negative predictive value is only about 64% (Ross et al., 2008). Therefore, negative biopsies are not particularly informative and do not aid in clinical decision-making (NCCN Pancreatic Adenocarcinoma Panel Members, 2007). Despite the availability of each of these tests, it is extremely difficult to accurately identify patients harboring a malignancy.

There are several consequences to this inherent difficulty in obtaining a confident diagnosis in lesions mimicking pancreatic cancer. Firstly, 7-16% (and as high as 25%) of patients who undergo a Whipple procedure or a radical pancreatectomy are found on final pathology to have benign lesions (van Heerden et al., 1981; Abraham et al., 2003; Kennedy et al., 2006; Hoshal et al., 2004; Aranha et al., 2003). These operations are extensive procedures associated with a high morbidity and a mortality rate. In the US, the overall in-hospital mortality rate for pancreatic resections is 7.6% (Meguid et al., 2008). An accurate serum test would help avoid such operations in patients with benign pancreatic disease.

Secondly, clinicians who encounter the non-specific signs associated with pancreatic cancer are often reluctant to refer the patient for a surgical opinion because they would like to avoid the morbidity of surgery if the patient has benign disease. This conservative, expectant approach may cause a delay in treatment that can result in the loss of any opportunity for potentially curative surgery. In recent surgical series, <20% of patients with pancreatic cancer have resectable disease (Li et al., 2004), although it is difficult to discern how many of those patients are found to have unresectable disease because of delays in diagnosis. As such, there remains a need for improved methods for making a definitive and early diagnosis of pancreatic cancer, thereby enabling a substantial impact on the outcomes of a significant proportion of patients.

Periampullary cancer. Pancreatic cancer may present with a biliary stricture or a pancreatic/periampullary mass. However, several benign entities and other periampullary adenocarcinomas can also present with strictures or masses that are difficult to accurately distinguish from pancreatic cancer using currently available technology.

The benign lesions include masses due to pancreatitis (acute, chronic, and autoimmune related), common bile duct strictures due to inflammatory or cholelithasis-related disease, and, less commonly, cysts (pseudocysts, simple cysts, and serous cystic neoplasms). Importantly, none of these lesions have malignant potential. If such lesions can be diagnosed confidently, they can be are treated nonsurgically, unless causing substantial symptoms. However, if a definitive benign diagnosis cannot be established on biopsy, surgical resection is the only remaining method of ruling out a pancreas cancer. Periampullary malignancies, including cancers of the duodenum, ampulla of Vater, and distal common bile duct, are often impossible to differentiate from pancreatic cancer using current radiologic and biopsy techniques. These other peripancreatic malignancies have a better prognosis than pancreatic cancer, suggesting different tumor biology and potentially the need for separate treatment approaches. However, at the moment, given the difficulty of accurate preoperative diagnosis, all are treated as if they are pancreatic cancer.

Presently, a clinician who encounters a patient with a pancreatic/periampullary mass or stricture must make decisions based on radiographic studies, serum tumor markers, and the availability of any tissue samples. Radiographic findings occur late in the course of pancreatic cancer, and findings are not specific. To date, no serum tumor marker has been shown to accurately and reliably diagnose pancreatic disease. Researchers have investigated various tumor antigens (e.g. CA 19-9), comparative proteomics techniques, and circulating levels of specific DNA and RNA molecules (e.g. mutant k-ras) but no single tumor marker has yet been validated as highly sensitive and specific for distinguising pancreatic cancer from benign pancreatic disease (Liang et al., 2009). Panels of markers are being investigated and show some promise, but require the combination of multiple laboratory techniques and may be difficult to implement.

Exocrine pancreas cancer treatments. Treatment of pancreatic cancer depends on the stage of the cancer. The Whipple procedure is the most common surgical treatment for cancers involving the head of the pancreas. This procedure involves removing the pancreatic head and the curve of the duodenum together (pancreato-duodenectomy), making a bypass for food from stomach to jejunum (gastro-jejunostomy) and attaching a loop of jejunum to the hepatic duct to drain bile (hepaticojejunostomy). It can be performed only if the patient is likely to survive major surgery and if the cancer is localized without invading local structures or metastasizing. It can, therefore, be performed in only the minority of cases.

Cancers of the tail of the pancreas can be resected using a procedure known as a distal pancreatectomy. Recently, localized cancers of the pancreas have been resected using minimally invasive (laparoscopic) approaches.

After surgery, adjuvant chemotherapy with gemcitabine or 5-fluorouracil has been shown in several large randomized studies to significantly increase the 5-year survival (from approximately 10 to 20%), and should be offered if the patient is fit after surgery. Addition of radiation therapy is a hotly debated topic, with groups in the US often favoring the use of adjuvant radiation therapy, while groups in Europe do not, due to the lack of any large randomized studies to show any survival benefit of this strategy.

Surgery can be performed for palliation, if the malignancy is invading or compressing the duodenum or colon. In that case, bypass surgery might overcome the obstruction and improve quality of life, but it is not intended as a cure.

In patients not suitable for resection with curative intent, palliative chemotherapy may be used to improve quality of life and gain a modest survival benefit. Gemcitabine was approved by the United States Food and Drug Administration in 1998, after a clinical trial reported improvements in quality of life and a 5-week improvement in median survival duration in patients with advanced pancreatic cancer. This marked the first FDA approval of a chemotherapy drug primarily for a nonsurvival clinical trial endpoint. Gemcitabine is administered intravenously on a weekly basis. xocrine pancreatic cancer (adenocarcinoma and less common variants) typically has a poor prognosis, partly because the cancer usually causes no symptoms early on, leading to locally advanced or metastatic disease at time of diagnosis.

Pancreatic cancer may occasionally result in diabetes. Insulin production is hampered, and it has been suggested the cancer can also prompt the onset of diabetes and vice versa. It can be associated with pain, fatigue, weight loss, jaundice, and weakness. Additional symptoms are discussed above.

For pancreatic cancer:
For all stages combined, the 1-year relative survival rate is 25%, and the 5-year survival is estimated as less than 5% to 6%.
For local disease, the 5-year survival is less than 20%.
For locally advanced and for metastatic disease, which collectively represent over 80% of individuals, the median survival is about 10 and 6 months,
respectively. Without active treatment, metastatic pancreatic cancer has a median survival of 3-5 months; complete remission is rare.

Outcomes with pancreatic endocrine tumors, many of which are benign and completely without clinical symptoms, are much better, as are outcomes with symptomatic benign tumors; even with actual pancreatic endocrine cancers, outcomes are rather better, but variable.

### II. METABOLOMIC MARKERS

As discussed in the Examples, the inventors have identified numerous metabolites that combine to create a metabalomic signature that distinguishes benign pancreatic masses and biliary strictures from adenocarcinoma (Tables 2, 3 and 5). The general methodology used to analyze metabolites includes Nuclear Magnetic Resonance and Gas Chromatography-Mass Spectrometry. These techniques are described generally below.

A. Nuclear Magnetic Resonance

Nuclear magnetic resonance spectroscopy, most commonly known as NMR spectroscopy, is a research technique that exploits the magnetic properties of certain atomic nuclei to determine physical and chemical properties of atoms or the molecules in which they are contained. It relies on the phenomenon of nuclear magnetic resonance and can provide detailed information about the structure, dynamics, reaction state, and chemical environment of molecules.

Most frequently, NMR spectroscopy is used by chemists and biochemists to investigate the properties of organic molecules, though it is applicable to any kind of sample that contains nuclei possessing spin. Suitable samples range from small compounds analyzed with 1-dimensional proton or carbon-13 NMR spectroscopy to large proteins or nucleic acids using 3 or 4-dimensional techniques. The impact of NMR spectroscopy on the sciences has been substantial because of the range of information and the diversity of samples, including solutions and solids.

When placed in a magnetic field, NMR active nuclei (such as ¹H or ¹³C) absorb electromagnetic radiation at a frequency characteristic of the isotope. The resonant frequency, energy of the absorption and the intensity of the signal are proportional to the strength of the magnetic field. For example, in a 21 tesla magnetic field, protons resonate at 900 MHz. It is common to refer to a 21 T magnet as a 900 MHz magnet, although different nuclei resonate at a different frequency at this field strength in proportion to their nuclear magnetic moments.

Depending on their local chemical environment, different nuclei in a molecule absorb at slightly different frequencies. Since this resonant frequency is directly proportional to the strength of the magnetic field, the shift is converted into a field-independent dimensionless value known as the chemical shift. The chemical shift is reported as a relative measure from some reference resonance frequency. (For the nuclei ¹H, ¹³C, and ²⁹Si, TMS (tetramethylsilane) is commonly used as a reference.) This difference between the frequency of the signal and the frequency of the reference is divided by frequency of the reference signal to give the chemical shift. The frequency shifts are extremely small in comparison to the fundamental NMR frequency. A typical frequency shift might be 100 Hz, compared to a fundamental NMR frequency of 100 MHz, so the chemical shift is generally expressed in parts per million (ppm). To detect such small frequency differences the applied magnetic field must be constant throughout the sample volume. High resolution NMR spectrometers use shims to adjust the homogeneity of the magnetic field to parts per billion (ppb) in a volume of a few cubic centimeters. In general, chemical shifts for protons are highly predictable since the shifts are primarily determined by simpler shielding effects (electron density), but the chemical shifts for many heavier nuclei are more strongly influenced by other factors including excited states (“paramagnetic” contribution to shielding tensor).

The chemical shift provides information about the structure of the molecule. The conversion of the raw data to this information is called assigning the spectrum. For example, for the ¹H-NMR spectrum for ethanol (CH₃CH₂OH), one would expect signals at each of three specific chemical shifts: one for the CH₃ group, one for the CH₂ group and one for the OH group. A typical CH₃ group has a shift around 1 ppm, a CH₂ attached to an OH has a shift of around 4 ppm and an OH has a shift around 2-3 ppm depending on the solvent used.

Because of molecular motion at room temperature, the three methyl protons average out during the course of the NMR experiment (which typically requires a few ms). These protons become degenerate and form a peak at the same chemical shift.

The shape and size of peaks are indicators of chemical structure too. In the example above—the proton spectrum of ethanol—the CH₃ peak would be three times as large as the OH. Similarly the CH₂ peak would be twice the size of the OH peak but only ⅔ the size of the CH₃ peak.

Software allows analysis of the size of peaks to understand how many protons give rise to the peak. This is known as integration—a mathematical process which calculates the area under a curve. The analyst must integrate the peak and not measure its height because the peaks also have width—and thus its size is dependent on its area not its height. However, it should be mentioned that the number of protons, or any other observed nucleus, is only proportional to the intensity, or the integral, of the NMR signal, in the very simplest one-dimensional NMR experiments. In more elaborate experiments, for instance, experiments typically used to obtain carbon-13 NMR spectra, the integral of the signals depends on the relaxation rate of the nucleus, and its scalar and dipolar coupling constants. Very often these factors are poorly known—therefore, the integral of the NMR signal is very difficult to interpret in more complicated NMR experiments.

Some of the most useful information for structure determination in a one-dimensional NMR spectrum comes from J-coupling or scalar coupling (a special case of spin-spin coupling) between NMR active nuclei. This coupling arises from the interaction of different spin states through the chemical bonds of a molecule and results in the splitting of NMR signals. These splitting patterns can be complex or simple and, likewise, can be straightforwardly interpretable or deceptive. This coupling provides detailed insight into the connectivity of atoms in a molecule.

Coupling to n equivalent (spin ½) nuclei splits the signal into a n+1 multiplet with intensity ratios following Pascal's triangle as described on the right. Coupling to additional spins will lead to further splittings of each component of the multiplet e.g. coupling to two different spin ½ nuclei with significantly different coupling constants will lead to a doublet of doublets (abbreviation: dd). Note that coupling between nuclei that are chemically equivalent (that is, have the same chemical shift) has no effect of the NMR spectra and couplings between nuclei that are distant (usually more than 3 bonds apart for protons in flexible molecules) are usually too small to cause observable splittings. Long-range couplings over more than three bonds can often be observed in cyclic and aromatic compounds, leading to more complex splitting patterns.

For example, in the proton spectrum for ethanol described above, the CH₃ group is split into a triplet with an intensity ratio of 1:2:1 by the two neighboring CH₂ protons. Similarly, the CH₂ is split into a quartet with an intensity ratio of 1:3:3:1 by the three neighboring CH₃ protons. In principle, the two CH₂ protons would also be split again into a doublet to form a doublet of quartets by the hydroxyl proton, but intermolecular exchange of the acidic hydroxyl proton often results in a loss of coupling information.

Coupling to any spin ½ nuclei such as phosphorus-31 or fluorine-19 works in this fashion (although the magnitudes of the coupling constants may be very different). But the splitting patterns differ from those described above for nuclei with spin greater than ½ because the spin quantum number has more than two possible values. For instance, coupling to deuterium (a spin 1 nucleus) splits the signal into a 1:1:1 triplet because the spin 1 has three spin states. Similarly, a spin 3/2 nucleus splits a signal into a 1:1:1:1 quartet and so on.

Coupling combined with the chemical shift (and the integration for protons) tells us not only about the chemical environment of the nuclei, but also the number of neighboring NMR active nuclei within the molecule. In more complex spectra with multiple peaks at similar chemical shifts or in spectra of nuclei other than hydrogen, coupling is often the only way to distinguish different nuclei.

The above description assumes that the coupling constant is small in comparison with the difference in NMR frequencies between the inequivalent spins. If the shift separation decreases (or the coupling strength increases), the multiplet intensity patterns are first distorted, and then become more complex and less easily analyzed (especially if more than two spins are involved). Intensification of some peaks in a multiplet is achieved at the expense of the remainder, which sometimes almost disappear in the background noise, although the integrated area under the peaks remains constant. In most high-field NMR, however, the distortions are usually modest and the characteristic distortions (roofing) can in fact help to identify related peaks.

Second-order effects decrease as the frequency difference between multiplets increases, so that high-field (i.e., high-frequency) NMR spectra display less distortion than lower frequency spectra. Early spectra at 60 MHz were more prone to distortion than spectra from later machines typically operating at frequencies at 200 MHz or above.

More subtle effects can occur if chemically equivalent spins (i.e., nuclei related by symmetry and so having the same NMR frequency) have different coupling relationships to external spins. Spins that are chemically equivalent but are not indistinguishable (based on their coupling relationships) are termed magnetically inequivalent. For example, the 4 H sites of 1,2-dichlorobenzene divide into two chemically equivalent pairs by symmetry, but an individual member of one of the pairs has different couplings to the spins making up the other pair. Magnetic inequivalence can lead to highly complex spectra which can only be analyzed by computational modeling. Such effects are more common in NMR spectra of aromatic and other non-flexible systems, while conformational averaging about C—C bonds in flexible molecules tends to equalize the couplings between protons on adjacent carbons, reducing problems with magnetic inequivalence.

B. Gas Chromatography-Mass Spectrometry

Gas chromatography-mass spectrometry (GC-MS) is a method that combines the features of gas-liquid chromatography and mass spectrometry to identify different substances within a test sample. The GC-MS is composed of two major building blocks: the gas chromatograph and the mass spectrometer. The gas chromatograph utilizes a capillary column which depends on the column's dimensions (length, diameter, film thickness) as well as the phase properties (e.g. 5% phenyl polysiloxane). The difference in the chemical properties between different molecules in a mixture will separate the molecules as the sample travels the length of the column. The molecules take different amounts of time (called the retention time) to come out of (elute from) the gas chromatograph, and this allows the mass spectrometer downstream to capture, ionize, accelerate, deflect, and detect the ionized molecules separately. The mass spectrometer does this by breaking each molecule into ionized fragments and detecting these fragments using their mass to charge ratio.

These two components, used together, allow a much finer degree of substance identification than either unit used separately. It is not possible to make an accurate identification of a particular molecule by gas chromatography or mass spectrometry alone. The mass spectrometry process normally requires a very pure sample while gas chromatography using a traditional detector (e.g., Flame Ionization Detector) detects multiple molecules that happen to take the same amount of time to travel through the column (i.e., have the same retention time) which results in two or more molecules to co-elute. Sometimes two different molecules can also have a similar pattern of ionized fragments in a mass spectrometer (mass spectrum). Combining the two processes reduces the possibility of error, as it is extremely unlikely that two different molecules will behave in the same way in both a gas chromatograph and a mass spectrometer. Therefore, when an identifying mass spectrum appears at a characteristic retention time in a GC-MS analysis, it typically lends to increased certainty that the analyte of interest is in the sample.

For the analysis of volatile compounds a Purge and Trap (P&T) concentrator system may be used to introduce samples. The target analytes are extracted and mixed with water and introduced into an airtight chamber. An inert gas such as Nitrogen (N₂) is bubbled through the water; this is known as purging. The volatile compounds move into the headspace above the water and are drawn along a pressure gradient (caused by the introduction of the purge gas) out of the chamber. The volatile compounds are drawn along a heated line onto a ‘trap’. The trap is a column of adsorbent material at ambient temperature that holds the compounds by returning them to the liquid phase. The trap is then heated and the sample compounds are introduced to the GC-MS column via a volatiles interface, which is a split inlet system. P&T GC-MS is particularly suited to volatile organic compounds (VOCs) and BTEX compounds (aromatic compounds associated with petroleum).

The most common type of mass spectrometer (MS) associated with a gas chromatograph (GC) is the quadrupole mass spectrometer, sometimes referred to by the Hewlett-Packard now Agilent) trade name “Mass Selective Detector” (MSD). Another relatively common detector is the ion trap mass spectrometer. Additionally one may find a magnetic sector mass spectrometer, however these particular instruments are expensive and bulky and not typically found in high-throughput service laboratories. Other detectors may be encountered such as time of flight (TOF), tandem quadrupoles (MS-MS) (see below), or in the case of an ion trap MSn where n indicates the number mass spectrometry stages.

A mass spectrometer is typically utilized in one of two ways: Full Scan or Selective Ion Monitoring (SIM). The typical GC-MS instrument is capable of performing both functions either individually or concomitantly, depending on the setup of the particular instrument. When collecting data in the full scan mode, a target range of mass fragments is determined and put into the instrument's method. An example of a typical broad range of mass fragments to monitor would be m/z 50 to m/z 400. The determination of what range to use is largely dictated by what one anticipates being in the sample while being cognizant of the solvent and other possible interferences. A MS should not be set to look for mass fragments too low or else one may detect air (found as m/z 28 due to nitrogen), carbon dioxide (m/z 44) or other possible interferences. Additionally if one is to use a large scan range then sensitivity of the instrument is decreased due to performing fewer scans per second since each scan will have to detect a wide range of mass fragments. Full scan is useful in determining unknown compounds in a sample. It provides more information than SIM when it comes to confirming or resolving compounds in a sample. During instrument method development it may be common to first analyze test solutions in full scan mode to determine the retention time and the mass fragment fingerprint before moving to a SIM instrument method.

In selected ion monitoring (SIM) certain ion fragments are entered into the instrument method and only those mass fragments are detected by the mass spectrometer. The advantages of SIM are that the detection limit is lower since the instrument is only looking at a small number of fragments (e.g., three fragments) during each scan. More scans can take place each second. Since only a few mass fragments of interest are being monitored, matrix interferences are typically lower. To additionally confirm the likelihood of a potentially positive result, it is relatively important to be sure that the ion ratios of the various mass fragments are comparable to a known reference standard.

After the molecules travel the length of the column, pass through the transfer line and enter into the mass spectrometer they are ionized by various methods with typically only one method being used at any given time. Once the sample is fragmented it will then be detected, usually by an electron multiplier diode, which essentially turns the ionized mass fragment into an electrical signal that is then detected. The ionization technique chosen is independent of using Full Scan or SIM.

By far the most common and perhaps standard form of ionization is electron ionization (EI). The molecules enter into the MS (the source is a quadrupole or the ion trap itself in an ion trap MS) where they are bombarded with free electrons emitted from a filament, not much unlike the filament one would find in a standard light bulb. The electrons bombard the molecules, causing the molecule to fragment in a characteristic and reproducible way. This “′hard ionization” technique results in the creation of more fragments of low mass to charge ratio (m/z) and few, if any, molecules approaching the molecular mass unit. Hard ionization is considered by mass spectrometrists as the employ of molecular electron bombardment, whereas “soft ionization” is charge by molecular collision with an introduced gas. The molecular fragmentation pattern is dependant upon the electron energy applied to the system, typically 70 eV (electron Volts). The use of 70 eV facilitates comparison of generated spectra with library spectra using manufacturer-supplied software or software developed by the National Institute of Standards (NIST-USA). Spectral library searches employ matching algorithms such as Probability Based Matching and dot-product matching that are used with methods of analysis written by many method standardization agencies. Sources of libraries include NIST, Wiley, the AAFS, and instrument manufacturers. Library coverage can be checked at Compound Search.

In chemical ionization a reagent gas, typically methane or ammonia is introduced into the mass spectrometer. Depending on the technique (positive CI or negative CI) chosen, this reagent gas will interact with the electrons and analyte and cause a ‘soft’ ionization of the molecule of interest. A softer ionization fragments the molecule to a lower degree than the hard ionization of EI. One of the main benefits of using chemical ionization is that a mass fragment closely corresponding to the molecular weight of the analyte of interest is produced.

In Positive Chemical Ionization (PCI) the reagent gas interacts with the target molecule, most often with a proton exchange. This produces the species in relatively high amounts. In Negative Chemical Ionization (NCI) the reagent gas decreases the impact of the free electrons on the target analyte. This decreased energy typically leaves the fragment in great supply.

When a second phase of mass fragmentation is added, for example using a second quadrupole in a quadrupole instrument, it is called tandem MS (MS/MS). MS/MS can sometimes be used to quantitate low levels of target compounds in the presence of a high sample matrix background. The first quadrupole (Q1) is connected with a collision cell (q2) and another quadrupole (Q3). Both quadrupoles can be used in scanning or static mode, depending on the type of MS/MS analysis being performed. Types of analysis include product ion scan, precursor ion scan, Selected Reaction Monitoring (SRM) (sometimes referred to as Multiple Reaction Monitoring (MRM)) and Neutral Loss Scan. For example: When Q1 is in static mode (looking at one mass only as in SIM), and Q3 is in scanning mode, one obtains a so-called product ion spectrum (also called “daughter spectrum”). From this spectrum, one can select a prominent product ion which can be the product ion for the chosen precursor ion. The pair is called a “transition” and forms the basis for SRM. SRM is highly specific and virtually eliminates matrix background.

### III. TREATMENT OF PANCREATIC CANCER

In some embodiments, the disclosure further provides treatment of pancreatic cancer. One of skill in the art will be aware of many treatments and treatment combinations may be used, some but not all of which are described below.

A. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present disclosure administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the disclosure, methods include identifying a patient in need of treatment. A patient may be identified, for example, based on taking a patient history or based on findings on clinical examination.

B. Cancer Treatments

1. Chemotherapy

A wide variety of chemotherapeutic agents may be used in accordance with the present disclosure. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethio-phosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (Abraxane®) and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, navelbine, farnesyl-protein tansferase inhibitors, transplatinum, 5-fluorouracil, vincristin, vinblastin and methotrexate and pharmaceutically acceptable salts, acids or derivatives of any of the above.

Of particular interest is FOLFIRINOX in treating various forms of advanced pancreatic cancer is a combination chemotherapy regimen made up of the following four drugs: folinic acid (leucovorin), a vitamin B derivative that modulates/potentiates/reduces the side effects of fluorouracil; fluorouracil (5-FU), a pyrimidine analog and antimetabolite which incorporates into the DNA molecule and stops DNA synthesis; irinotecan (Camptosar), a topoisomerase inhibitor, which prevents DNA from uncoiling and duplicating; and oxaliplatin (Eloxatin), a platinum-based antineoplastic agent, which inhibits DNA repair and/or DNA synthesis. The regimen emerged in 2010 as a new treatment for patients with metastatic pancreatic cancer

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p9′7), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the gene therapy may be applied to the subject. Suitable genes included inducers of cellular proliferation, tumor suppressors, or regulators of programmed cell death.

6. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DRS/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

C. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regards, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### IV. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Serum Samples. Venous blood samples were obtained from 157 patients who had a pancreatic or periampullary lesion on diagnostic imaging. All patients provided informed consent for study participation and the study was approved by the Conjoint Health Research Ethics Board at the University of Calgary (IRB #E20846). All patients were fasting for at least 8 hours at the time of sample collection.

For patients not undergoing surgical resection, samples were collected at a licensed laboratory collection facility. For patients undergoing surgical resection, samples were collected on the day of surgery, prior to any surgical manipulation. Serum samples were collected and stored as previously described (Bathe et al., 2011).

Patient Data. For all patients, clinical data were collected prospectively as part of the serum banking process, using standardized forms. Each patient was classified as having either a malignant or a benign pancreatic/periampullary lesion based on review of pathology, diagnostic imaging, and operative and clinic notes. Malignancies included pancreatic, distal bile duct, ampullary and duodenal adenocarcinomas (all residing in the pancreatic and periampullary regions). In cases where a definitive pathologic diagnosis was not available because no resection occurred (n=65), the lesion was classified according to the diagnosis favoured by the consulting surgeon based on the clinical course of the patient. For malignant lesions, stage classification was assigned according to the American Joint Committee on Cancer (AJCC) Cancer Staging Manual (7th Edition) (Edge et al., 2010).

Metabolomic Analysis. Serum samples for this study were analysed using ¹H-NMR spectroscopy and GC-MS according to previously published protocols (Bathe et al., 2011; Farshidfar et al., 2012). For GC-MS, each sample was randomly assigned to one of four sequential days for extraction, and then, with a separate randomization, assigned to one of four sequential days for derivatization and GC-MS analysis.

Metabolites from the ¹H-NMR spectroscopy dataset were identified and quantified using the Human Metabolome Database (HMDB, version 2.5) (Wishart et al., 2009) and Chenomx NMR Suite 6.1 software (Chenomx Inc., Edmonton, Canada) using the ‘Targeted Profiling’ approach (Weljie et al., 2006). Metabolites and features from the GC-MS dataset were identified using Metabolite Detector software (Hiller et al., 2009) (Version 2.06, Technische Universitat Carolo-Wilhelmina zu Braunschweig, Braunschweig, Germany) and the GOLM metabolite database (Kopka et al., 2005). All metabolite features, whether matched to an identification by MetaboliteDetector or not, were included in the dataset for further analysis. Species matched to an entity in the GOLM database that does not yet have an associated chemical name were labelled with the word “Match,” their RI value, and the list of m/z values for quantified ions (e.g., “Match: RI 1416.54, Ions 110 134 184 217 228”); and species not matched to any entity in the GOLM database were labelled with the word “Unmatched,” their RI value, and the list of m/z values for quantified ions (e.g., “Unmatched: RI 2475.33, Ions 73 375 376”).

Data pre-processing. All zero values were considered as missing values and all metabolites or features with >50% missing values were excluded from further analysis. The resulting ¹H-NMR dataset contained 60 metabolites and the GC-MS dataset contained 123 metabolites/features for further analysis. Data pre-processing was conducted separately for the ¹H-NMR spectroscopy and GC-MS datasets using STATA (version 12.0, StataCorp, College Station, Texas) and consisted of: median fold change normalization (Veselkov et al., 2011); logarithmic transformation; centering; and unit variance (van den Berg et al., 2006).

The resulting datasets had 22 metabolites in common (alanine, aspartate, citrate, glucose, glutamate, glutamine, glycerol, glycine, histidine, hypoxanthine, isoleucine, methionine, ornithine, phenylalanine, proline, pyroglutamate, serine, threonine, tryptophan, tyrosine, urea, and valine). For each of these metabolites, the mean of ¹H-NMR spectroscopy and GC-MS values was calculated and included in a new Combined dataset as previously reported (Booth et al., 2011). To this was added the remaining 38 non-shared ¹H-NMR metabolites and 101 non-shared GC-MS metabolites/features, giving a total of 161 metabolites/features in the Combined dataset.

Multivariate projection modeling. Three random allocations of the 157 patient samples to training and test sets were conducted, in a 50:50 split, with stratification for diagnosis (malignant vs. benign), serum sampling year (<2008 vs. >2009), GC-MS extraction day (1 or 2 vs. 3 or 4), and GC-MS derivatization day (1 or 2 vs. 3 or 4).

SIMCA-P+ (Version 12.0, Umetrics, Umea, Sweden) software was used for all multivariate projection modeling. All modeling procedures were conducted separately for each of the three training sets, using the ¹H-NMR spectroscopy, GC-MS, and Combined datasets. Thus, a total of 9 training models were generated (3 datasets×3 trials). For each model, metabolites were pre-filtered using a t-test of distributions between malignant and benign lesions (p-value <0.3). Unsupervised principal component analysis (PCA) was then conducted to look for marked outliers and any latent structures within each model (Trygg et al., 2007).

For each of the 9 training sets, bidirectional orthogonal partial least squares (O2PLS) analysis was conducted using the following covariates: patient age, gender, lesion location, lesion type, surgical resection, cancer staging (where applicable), jaundice, diabetes mellitus, bowel cleansing, sampling year, and sampling location. For analysis of the GC-MS and Combined datasets, extraction and derivatization days were added as covariates. Metabolites contributing more to the modeling of non-diagnostic covariates than to the modeling of the diagnostic class, were excluded iteratively until diagnostic class was the covariate contributing most to the overall model. The resulting reduced list of metabolites was then submitted to orthogonal partial least squares-discriminant analysis (OPLS-DA) modeling. Using Variable Importance to Projection (VIP) values and coefficients, the list of metabolites was iteratively reduced to the absolute minimum required to maintain the strength of model parameters. For each training set, the resulting focused metabolite list was compiled and model parameters reported. The validity of the generated models was then tested by prediction of the diagnostic classification in the respective independent test sets, and area under the receiver operating curve (AUROC) values were calculated.

Metabolic pathway analysis. The focused list of metabolites from each trial was extracted, along with their respective regression coefficients and VIP values. These lists were combined for the three trials for each dataset. For metabolites found in the focused list for more than one trial, mean coefficient and VIP values were calculated. This yielded a focused list of metabolites for ¹H-NMR spectroscopy, GC-MS, and Combined datasets, respectively. A list of consistently contributing metabolites across all trials and datasets was compiled and submitted for topological metabolic pathway analysis using MetaboAnalyst software (version 2.0, Metabolomics Innovation Centre, Edmonton, Alberta) (Xia and Wishart, 2010; 2011). Where a metabolite was contributing to all three datasets, the average concentration/intensity data from the Combined dataset was used. Otherwise, the concentration/intensity data from the ¹H-NMR spectroscopy or GC-MS dataset was used, as appropriate.

### Example 2

### Results

Demographic and technical factors. For each of the three separate randomized allocations to the 50:50 split, the training group contained 80 patient samples, and the test group contained 77 patient samples. Clinical and technical factors appeared evenly distributed for each allocation (Table 1).

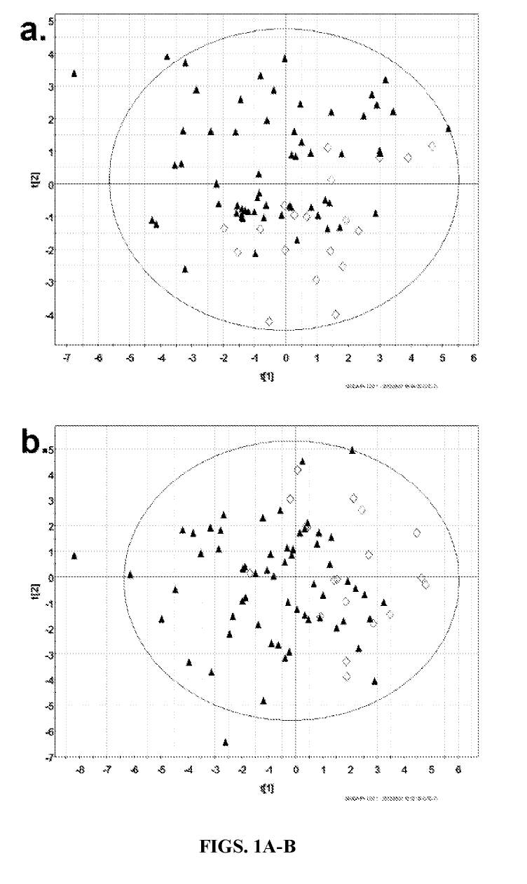

Principal component analysis. On PCA modeling, no marked latent structures were identified and no sample was a consistent outlier across allocation trials. Several models showed some degree of separation between malignant and benign samples in component 1 or 2 (FIGS. 1A-B).

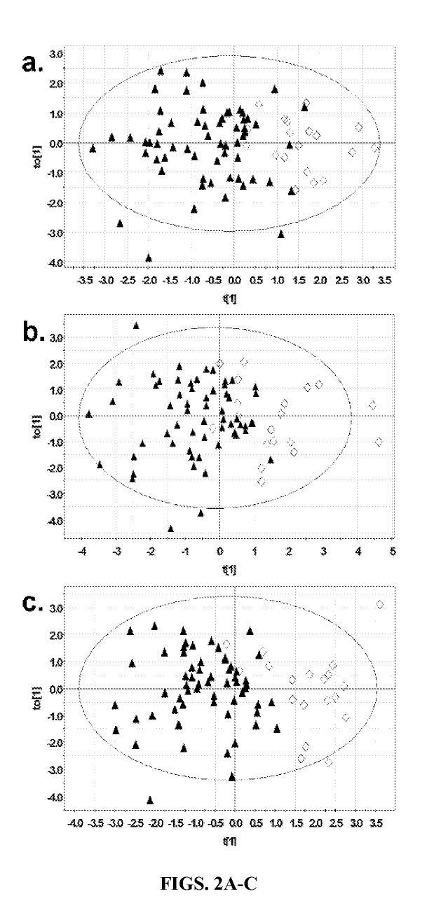

Orthogonal multivariate projection modeling. Table 2 summarizes the results of modeling for the ¹H-NMR spectroscopy, GC-MS and Combined datasets, and FIGS. 2A-C display the respective scores plots. These results indicate the ability of metabolites from these three datasets to distinguish malignant versus benign lesions in training sets of 80 patient samples, with independent validation in test sets of 77 patient samples.

For the ¹H-NMR spectroscopy dataset, the focused metabolite lists contained an average of 14 metabolites and the resulting models had the following average parameters: R²Y 0.308, Q² 0.184, and CV-ANOVA p value 1.8×10⁻³. On independent validation in the test sets, the average AUROC was 0.74 (SE=0.06). When the same training sets were tested in the GC-MS dataset, the average focused metabolite list contained 18 metabolites/features. Average model parameters were R²Y 0.312, Q² 0.188, and CV-ANOVA p value 8.4×10⁻⁴. On independent validation in the respective test sets, the mean AUROC was 0.62 (SE=0.08). For the Combined dataset, focused metabolite lists contained, on average, 20 metabolites and the resulting models had, on average, R²Y 0.478, Q² 0.324, and CV-ANOVA p value 6.14×10⁻⁶. On validation in the respective test sets, the average AUROC was 0.66 (SE=0.08).

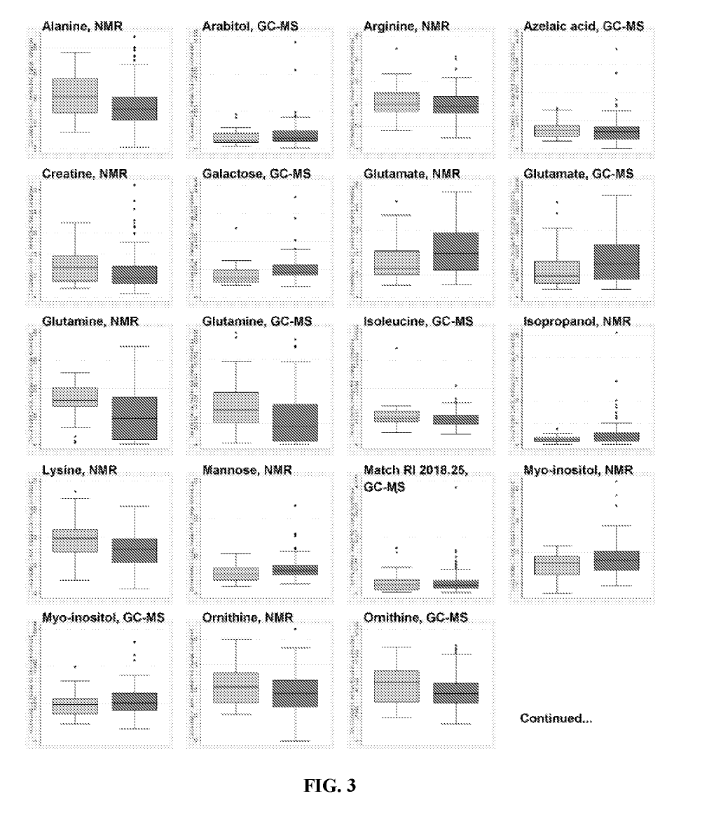

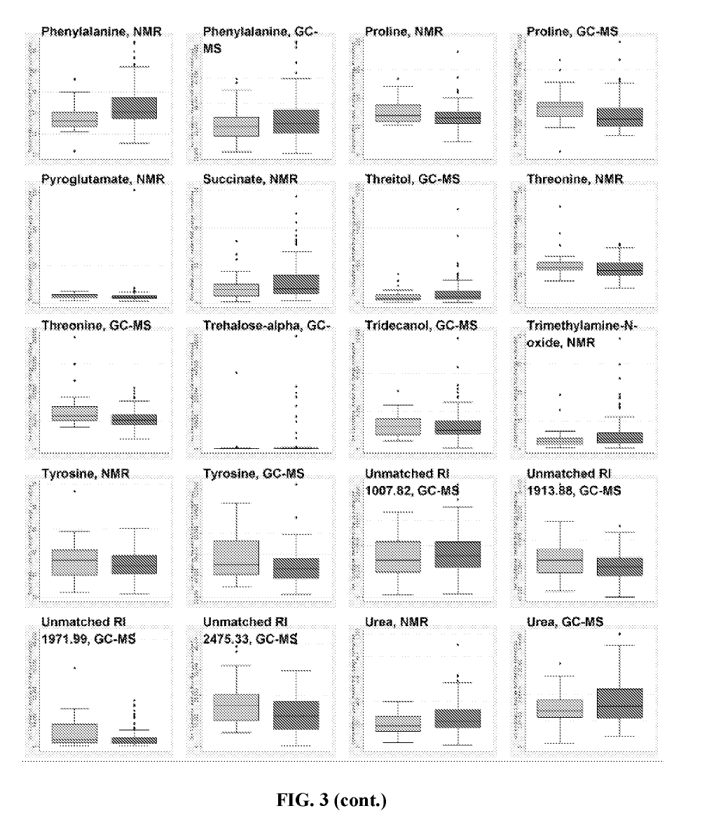

Eight metabolites were found to consistently contribute to the malignant/benign profile across all three datasets (Table 3): higher levels of glutamate, myo-inositol, phenylalanine, and urea were consistently correlated with malignancy; while higher levels of glutamine, ornithine, proline, and threonine were consistently correlated with benign disease. An additional 22 metabolites were found less consistently across the datasets. These metabolites were identified in the modeling for at least 2 trials in at least one dataset, or in different trials in different datasets. Of these, 9 metabolites (non-italicized in Table 3) were associated with malignancy and 13 metabolites were associated with benign disease (Table 3, non-italicized). Whisker plots of the raw data for all 30 consistently contributing metabolites are shown in FIG. 3.

Metabolic pathway analysis. The predominant differences between malignant and benign patient samples appeared to occur within amino acid and carbohydrate metabolic pathways (Table 4). The arginine/proline pathway had the largest impact factor (0.456, p=0.000085) with consistently higher levels of arginine, creatine, glutamine, ornithine, and proline seen in the benign samples and consistently higher levels of glutamate and urea in malignant samples. The alanine/aspartate/glutamate pathway had the next largest impact factor (0.441, p=0.00026), reflecting the consistently higher levels of alanine and glutamine in benign samples versus glutamate and succinate in malignant samples. Galactose levels were higher in malignant samples and the galactose metabolism pathway had the third largest impact factor (0.224, p=0.000086). The list of all statistically impacted pathways is included in Table 4.

### Example 3

### Discussion

Focused metabolomic profiles, containing as few as 14-18 metabolites, can discriminate between serum samples from patients with malignant versus benign pancreatic/periampullary lesions. The training set, these focused metabolomic profiles produced OPLS-DA models with R² values of 0.30-0.48, indicating that 30-48% of the observed variance in metabolite levels was attributable to the diagnostic classification. These values are in the range expected for clinical specimens (Fiehn et al., 2010), are in keeping with the clustering of samples by diagnostic category seen in the first and second components of unsupervised PCA, and were sufficient to statistically discriminate between diagnostic classes as indicated by the CVANOVA p-values.

The metabolomic profile of malignant versus benign lesions was validated in separate test sets with AUROC values of 0.62-0.74. This level of performance is similar to that of the widely used serum tumor marker CA 19-9, suggesting that the metabolomic profile may have clinical utility (Goonetilleke et al., 2007). Further prospective validation will be required to test its true utility in clinical decision-making, where it would be combined with other available data, including CA 19-9.

In addition to its potential clinical utility, the developed metabolomic profile also offers insights into the metabolomic pathways altered in patients with a pancreatic/periampullary malignancy. The results clearly indicate a tipping of the balance of amino acid metabolism towards higher glutamate levels in malignant samples and higher glutamine and alanine levels in benign disease. These observations are consistent with earlier findings published by the inventors, which found elevated levels of glutamate in the serum of pancreatic cancer patients when compared to that of patients with benign pancreatic or biliary disease (Bathe et al., 2011). It is also consistent with findings in many cancer model systems that show a switch to glutamine consumption, and increased glutamate and succinate production, in patients with rapidly proliferating cancer cells, the “Warburg effect” (Weljie et al., 2011; Morvan et al., 2007).

Arginine and ornithine are part of the urea cycle and feed the production of putrescine, the rate-limiting step in protein synthesis. The conversion of arginine to ornithine, by the enzyme arginase, has been suggested as a major regulator of cell growth (Weljie et al., 2011). It is therefore interesting that arginine and ornithine levels were lower in the serum of patients with pancreatic cancer compared to benign pancreatic lesions. The level of urea, a side product of arginine-to-ornithine conversion, was slightly higher in patients with pancreatic cancer. Together, these findings suggest altered arginase activity in patients with pancreatic and periampullary adenocarcinomas.

The correlation of serum galactose with pancreatic cancer is seen in the GC-MS dataset only, although NMR with the utilized settings may not detect galactose. A similar relationship was recently observed with colorectal cancer patients (Farshidfar et al., 2012), but further investigation is needed before any putative mechanism could be proposed.

Overall, the models performed slightly better than the GC-MS models, with a smaller standard error for the AUROC values. In addition, the average standard error for the metabolite coefficients was 62% higher for GC-MS compared to ¹H-NMR spectroscopy, indicating more variability in the regression modeling of metabolites in the GC-MS model. Finally, the consistency of metabolites identified across allocation sets was higher for ¹H-NMR than for GC-MS. For ¹H-NMR, 58% of metabolites important to the final focused list were identified in 2 or more of the allocation sets, while the same proportion for the GC-MS dataset was only 36% (p=0.04). This suggests that the distinguishing metabolite set detectable by ¹H-NMR spectroscopy was more stable from patient to patient than that identified by GC-MS.

The method of creating and modeling a Combined ¹H-NMR spectroscopy/GC-MS dataset implemented in this study did not produce models that were stronger than either of the two platforms individually. The inventors had hypothesized that this approach could harness the relative strengths of each platform, providing stronger predictions. However, the Combined dataset models performed only slightly better (average AUROC 0.66±0.08) than the GC-MS models (0.62±0.08) and not as well as the ¹H-NMR models (0.74±0.06).

When the Combined dataset was constructed, a simple averaging technique was used for combining metabolites detected by both platforms. Other approaches to combining data are being developed and used around the world, but no standard approach has yet been established (Maher et al., 2011; Gu et al., 2011). Studies combining other types of quantitative metabolomics data have also failed to show strong improvements in performance over since source approaches (Schicho et al., 2012). Further work in this field may result in a method that effectively capitalizes on the relative strengths of multiplatform detection, to produce an even stronger model and profile of metabolites that are diagnostically predictive.

The main limitations of this work are related to the current performance of ¹H-NMR spectroscopy and GC-MS platforms, the technique used for combining data from the two platforms, the nature of the patients included in the study, and the need for external validation. Techniques for optimizing and expanding the metabolome detection abilities of ¹H-NMR spectroscopy and GC-MS are continually emerging. The current results are based on the best knowledge and methods available at this time, but future studies with improved techniques may detect additional or different metabolites that are even more strongly correlated and predictive of the difference between serum samples in patients with malignant and benign pancreatic lesions.

Given the exploratory nature of this work, patients with a range of pancreatic/periampullary adenocarcinomas were included. Their clinical characteristics were thus quite heterogeneous. In particular, 19 patients had known metastatic disease, which may impact the applicability of the currently developed metabolite profiles to patients with non-metastatic disease. To address this issue, ongoing work aims to further refine the proposed profile using metabolomic data from patients who had resectable disease.

Finally, the metabolomic profiles that separate malignant from benign lesions were developed using training sets of 80 patients each, and tested for validity in an independent set of 77 patients. Further validation using an external cohort of patients (separate sampling time, storage time, laboratory preparation, platform analysis, and data processing) is needed to fully validate the proposed metabolomic profile. Nevertheless, the robust detection of multiple markers across multiple detection modalities provides strong evidence that serum metabolic profiles have potential utility in clinical management.

TABLE 1
Clinical and technical variables for each allocation of training and test sets

| | | Allocation A | | | Allocation B | | | Allocation C | | |
|---|---|---|---|---|---|---|---|---|---|---|
| | | Training | Test | | Training | Test | | Training | Test | |
| | | N = 80 | N = 77 | p* | N = 80 | N = 77 | p | N = 80 | N = 77 | p* |
| Age | <60 yrs | 24 | 24 | 0.87 | 27 | 21 | 0.38 | 22 | 26 | 0.40 |
| | ≥60 yrs | 56 | 53 | | 53 | 56 | | 58 | 51 | |
| Gender | Male | 45 | 37 | 0.31 | 46 | 36 | 0.18 | 40 | 42 | 0.57 |
| | Female | 35 | 40 | | 34 | 41 | | 40 | 35 | |
| Lesion location | Head/uncinate | 52 | 58 | 0.31 | 54 | 56 | 0.59 | 55 | 55 | 0.46 |
| | Body/tail | 20 | 15 | | 19 | 16 | | 20 | 15 | |
| Lesion type | Mass | 58 | 53 | 0.37 | 59 | 52 | 0.57 | 54 | 57 | 0.12 |
| | Stricture | 8 | 11 | | 7 | 12 | | 13 | 6 | |
| | Cyst | 9 | 11 | | 11 | 9 | | 12 | 8 | |
| Diagnosis | Malignant | 61 | 61 | 0.61 | 61 | 61 | 0.61 | 61 | 61 | 0.61 |
| | Benign | 19 | 16 | | 19 | 16 | | 19 | 16 | |
| Stage (for | I | 11 | 8 | 0.39 | 5 | 14 | 0.24 | 12 | 7 | 0.25 |
| malignant | II | 27 | 28 | | 30 | 25 | | 27 | 28 | |
| lesions only) | III | 16 | 13 | | 18 | 11 | | 14 | 15 | |
| | IV | 7 | 12 | | 8 | 11 | | 8 | 11 | |
| Surgically | Yes | 48 | 44 | 0.72 | 43 | 49 | 0.21 | 49 | 43 | 0.49 |
| resected | No | 32 | 33 | | 37 | 28 | | 31 | 34 | |
| Jaundice | Yes | 13 | 18 | 0.26 | 14 | 17 | 0.47 | 15 | 16 | 0.75 |
| | No | 67 | 59 | | 66 | 60 | | 65 | 61 | |
| Diabetes | Yes | 20 | 13 | 0.21 | 17 | 16 | 0.94 | 18 | 15 | 0.64 |
| mellitus | No | 60 | 64 | | 63 | 61 | | 62 | 62 | |
| Bowel | Yes | 43 | 43 | 1.0 | 42 | 44 | 0.30 | 47 | 39 | 0.46 |
| cleansing | No | 25 | 25 | | 29 | 21 | | 24 | 26 | |
| Sampling year | 2006-8 | 45 | 44 | 0.91 | 45 | 44 | 0.91 | 45 | 44 | 0.91 |
| | 2009-10 | 35 | 33 | | 35 | 33 | | 35 | 33 | |
| Sampling | Laboratory | 12 | 17 | 0.25 | 17 | 12 | 0.36 | 11 | 18 | 0.12 |
| location | OR | 68 | 60 | | 63 | 65 | | 69 | 59 | |
| GC-MS | Day 1/2 | 42 | 38 | 0.69 | 42 | 38 | 0.69 | 42 | 38 | 0.69 |
| extraction | Day 3/4 | 38 | 39 | | 38 | 39 | | 38 | 39 | |
| GC-MS | Day 1/2 | 40 | 41 | 0.68 | 40 | 41 | 0.68 | 40 | 41 | 0.68 |
| derivatization | Day 3/4 | 40 | 36 | | 40 | 36 | | 40 | 36 | |

*P values are for Mann-U-Witney testing between subgroups.
Italicized variables were used as stratification factors in the randomized allocation process.

TABLE 2
Results of orthogonal partial least squares discriminant analysis
(OPLS-DA)

| | Mean of training sets | | | | Mean of test sets | |
|---|---|---|---|---|---|---|
| | (n = 80 each) | | | | (n = 77 each) | |
| Dataset | X | R² | Q² | p | AUROC | SE |
| ¹H-NMR | 14 | 0.308 | 0.184 | 1.80 × 10⁻³ | 0.74 | 0.06 |
| GC-MS | 18 | 0.312 | 0.188 | 8.40 × 10⁻⁴ | 0.62 | 0.08 |
| Combined* | 20 | 0.478 | 0.324 | 6.14 × 10⁻⁶ | 0.66 | 0.08 |

*The Combined dataset includes metabolite features from both ¹H-NMR and GC-MS data, with averaged values for metabolites detected by both platforms.
X: Mean number of unique metabolites/features in the focused metabolite lists across three randomized allocations of training/test set assignment
R²: goodness of fit
Q²: predictive ability of model (7-fold internal cross validation)
p: p-value for CVANOVA testing
AUROC: area under the receiver operating curve
SE: standard error

TABLE 3
Summary list of metabolite features included in final focused models

| | | | Mean | Mean SE | Mean | Mean SE |
|---|---|---|---|---|---|---|
| | Metabolite | Datasets | Coeff | (Coeff) | VIP | (VIP) |
| Higher in Malignant | Galactose | G, C | 0.121 | 0.069 | 1.123 | 0.683 |
| | Unmatched RI: 1007.82 QI: 67, 82, | G, C | 0.120 | 0.074 | 1.337 | 0.708 |
| | 83 | | | | | |
| | Isopropanol | N, C | 0.114 | 0.042 | 1.001 | 0.382 |
| | Phenylalanine | N, G, C | 0.109 | 0.057 | 1.052 | 0.621 |
| | Glutamate | N, G, C | 0.105 | 0.064 | 1.127 | 0.616 |
| | Mannose | N, C | 0.102 | 0.069 | 1.220 | 0.410 |
| | Trimethylamine-N-oxide | N | 0.092 | 0.061 | 0.867 | 0.503 |
| | Arabitol | G, C | 0.090 | 0.047 | 0.967 | 0.409 |
| | Threitol | G, C | 0.088 | 0.080 | 0.889 | 0.816 |
| | Succinate | N, C | 0.086 | 0.115 | 0.743 | 0.777 |
| | Urea | N, G, C | 0.074 | 0.058 | 0.965 | 0.604 |
| | Myo-Inositol | N, G, C | 0.070 | 0.061 | 0.991 | 0.582 |
| | Trehalose-alpha | G, C | 0.059 | 0.053 | 0.624 | 0.572 |
| Higher in Benign | Match RI: 2018.25 QI: 191, 217, | G, C | −0.029 | 0.055 | 0.568 | 0.680 |
| | 305, 318, 507 | | | | | |
| | Tridecanol | G | −0.060 | 0.051 | 0.738 | 0.613 |
| | Azelaic acid | G | −0.061 | 0.038 | 0.814 | 0.526 |
| | Unmatched RI: 2475.33 QI: 73, | G, C | −0.066 | 0.048 | 0.791 | 0.475 |
| | 375, 376 | | | | | |
| | Pyroglutamate | N | −0.068 | 0.036 | 0.696 | 0.306 |
| | Isoleucine | G | −0.069 | 0.091 | 0.778 | 1.069 |
| | Tyrosine | N, G | −0.074 | 0.058 | 0.862 | 0.669 |
| | Arginine | N, C | −0.080 | 0.055 | 0.721 | 0.500 |
| | Unmatched RI: 1913.88 QI: 156, | G, C | −0.090 | 0.067 | 1.092 | 0.863 |
| | 174, 317 | | | | | |
| | Proline | N, G, C | −0.096 | 0.063 | 1.009 | 0.547 |
| | Alanine | N, C | −0.098 | 0.041 | 0.853 | 0.311 |
| | Ornithine | N, G, C | −0.104 | 0.068 | 0.997 | 0.687 |
| | Creatine | N, C | −0.107 | 0.041 | 0.952 | 0.267 |
| | Glutamine | N, G, C | −0.115 | 0.072 | 1.107 | 0.686 |
| | Lysine | N, C | −0.117 | 0.037 | 1.289 | 0.345 |
| | Threonine | N, G, C | −0.137 | 0.065 | 1.360 | 0.538 |
| | Unmatched RI: 1971.99 QI: 185, | G, C | −0.138 | 0.069 | 1.346 | 0.640 |
| | 247, 275 | | | | | |

N: ¹H-nuclear magnetic resonance spectroscopy,
G: gas chromatography mass spectrometry,
C: combined dataset,
Coeff: regression coefficient for given X variable (metabolite) in the modeled Y variable (malignant versus benign), positive values associated with malignancy and negative values associated with benign disease;
SE: standard error; RI: retention index,
QI: quantification ions;
VIP: variable importance to projection expresses overall contribution to the model.
Metabolite features in italics were found in the focused lists for all three datasets.

TABLE 4
Topological metabolic pathway analysis

| | Total | Hits in | | |
|---|---|---|---|---|
| | Compounds | Current | | Impact |
| Metabolic Pathway | in Pathway | Dataset | p | factor |
| Arginine and proline | 77 | 7 | 8.49E−05 | 0.456 |
| metabolism | | | | |
| Alanine, aspartate and | 24 | 4 | 2.60E−04 | 0.441 |
| glutamate metabolism | | | | |
| Galactose metabolism | 41 | 3 | 8.63E−05 | 0.224 |
| Lysine degradation | 47 | 1 | 4.09E−03 | 0.147 |
| D-Glutamine and D- | 11 | 2 | 1.37E−03 | 0.139 |
| glutamate metabolism | | | | |
| Inositol phosphate | 39 | 1 | 3.00E−02 | 0.137 |
| metabolism | | | | |
| Phenylalanine metabolism | 45 | 3 | 6.60E−03 | 0.119 |
| Aminoacyl-tRNA | 75 | 10 | 8.90E−07 | 0.113 |
| biosynthesis | | | | |
| Lysine biosynthesis | 32 | 1 | 4.09E−03 | 0.100 |
| Glycine, serine and | 48 | 2 | 7.07E−04 | 0.097 |
| threonine metabolism | | | | |
| Tyrosine metabolism | 76 | 2 | 2.77E−02 | 0.047 |
| Taurine and hypotaurine | 20 | 1 | 8.27E−03 | 0.032 |
| metabolism | | | | |
| Fructose and mannose | 48 | 1 | 1.56E−03 | 0.029 |
| metabolism | | | | |
| Butanoate metabolism | 40 | 2 | 6.28E−03 | 0.018 |
| Valine, leucine and | 27 | 2 | 9.74E−04 | 0.013 |
| isoleucine biosynthesis | | | | |
| Glutathione metabolism | 38 | 3 | 3.35E−03 | 0.013 |
| Phenylalanine, tyrosine and | 27 | 2 | 1.05E−02 | 0.008 |
| tryptophan biosynthesis | | | | |
| Purine metabolism | 92 | 2 | 5.70E−04 | 0.008 |

Produced using MetaboAnalyst software.
For each pathway, the total number of known metabolites, along with the number of those found in the current dataset (“hits”) are reported.
The p value is reported for the statistical comparison of metabolite feature levels between malignant and benign samples.
The impact factor expresses the degree of centrality of the identified changes to the pathway functioning overall.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Abraham et al., Am. J. Surg. Pathol., 27:110-120, 2003.
Aranha et al., J. Am. Coll. Surg., 197:223-231, 2003.
Asiago et al., Cancer Res., 8309-8318, 2010.
Austin-Ward and Villaseca, Revista Medica de Chile, 126(7):838-845, 1998.
Bathe et al., Cancer Epidemiol Biomarkers Prey 20:140-7, 2011.
Begley et al., Anal. Chem., 81(16):7038-7046, 2009.
Booth et al., J Proteome Res 10:3190-9, 2011.
Bukowski et al., Clinical Cancer Res., 4(10):2337-2347, 1998.
Camp and Vogel, Am Surg 70:6-12, 2004.
Chari et al., Gastroenterol., 129:504-511, 2005.
Chari et al., Gastroenterol., 134:95-101, 2008.
Christodoulides et al., Microbiology, 144(Pt 11):3027-3037, 1998.
Davidson et al., J. Immunother 21(5):389-398, 1998.
Dunn et al., Metabolomics, 3(4):413-426, 2007.
Edge et al., ed., AJCC Cancer Staging Manual. 7th ed. New York: Springer-Verlag, 2010.
Fang et al., Cancer Sci., 98:1678-1682, 2007.
Farshidfar et al., Genome Med 4:42, 2012.
Fiehn et al., PloS One, 5(12):e15234, 2010.
Fogel et al., Gastrointest. Endosc., 63:71-77, 2006.
Goonetilleke and Siriwardena, Eur J Surg Oncol 33:266-70, 2007.
Gu et al., Anal Chim Acta 686:57-63, 2011.
Hanibuchi et al., Int. J Cancer, 78(4):480-485, 1998.
Hellstrand et al., Acta Oncologica, 37(4):347-353, 1998.
Hiller et al., Anal Chem 81:3429-39, 2009.
Hoshal Jr. et al., Am. Surg., 70:121-125, 2004.
Hui and Hashimoto, Infection Immun., 66(11):5329-5336, 1998.
Ju et al., Gene Ther., 7(19):1672-1679, 2000.
Kennedy et al., Am. J. Surg., 191:437-441, 2006.
Kinney T., Surg Clin North Am 90:235-49, 2010.
Kopka et al., Bioinformatics, (England), 21:1635-1638, 2005.
Larsen, Dan. Med. Bull., 40:153-162, 1993.
Li et al., Lancet., 363:1049-1057, 2004.
Liang et al., Intl. I Clinical Experi. Pathol., 2(1):1-10, 2009.
Maher et al., J Proteome Res 10:1737-45, 2011.
Mahmoudi et al., World J Gastroenterol., 14:569-573, 2008.
Meguid et al., J. Am. Coll. Surg., 206:622 el-9, 2008.
Meisterfeld et al., Exp. Clin. Endocrinol. Diabetes, 116(Suppl 1):S7-S12, 2008.
Mitchell et al., Ann. NY Acad Sci., 690:153-166, 1993.
Mitchell et al., J. Clin. Oncol., 8(5):856-869, 1990.
Morton et al., Arch. Surg., 127:392-399, 1992.
Morvan D, Demidem A, Cancer Res 67:2150-9, 2007.
NCCN Pancreatic Adenocarcinoma Panel Members., NCCN clinical practice guidelines in oncology: pancreatic adenocarcinoma, Version 1.2008, Natl. Comprehen. Cancer Network, Inc, 2007.
Nicholson et al., Xenobiotica, 29:1181-1189, 1999.
Nishijima et al., Jpn. J. Clin. Oncol., 27:13-17, 1997.
Pannala et al., Gastroenterol., 134:981-987, 2008.
Pietras et al., Oncogene, 17(17):2235-2249, 1998.
Qin et al., Proc. Natl. Acad. Sci. USA, 95(24):14411-14416, 1998.
Ravindranath and Morton, Intern. Rev. Immunol., 7: 303-329, 1991.
Ritchie et al., BMC Med, 8:13, 2010.
Rosenberg et al., Ann. Surg. 210(4):474-548, 1989.
Rosenberg et al., N Engl. I Med., 319:1676, 1988.
Ross et al., Gastrointest. Endosc., 68:461-466, 2008.
Savides et al., Gastrointest. Endosc., 66:277-282, 2007.
Schicho et al., J Proteome Res 11:3344-57, 2012.
Simons et al., J Gastrointest Surg 13:1798-805, 2009.
Simunovic et al., Ann Surg Oncol 17:2537-44, 2010.
Sugimoto et al., Metabolomics, 6:78-95, 2010.
Trygg et al., Anal. Chem., 77(24):8086-8094, 2005.
Trygg et al., J. Proteome Res., 6:469-479, 2007.
U.S. Pat. No. 5,739,169
U.S. Pat. No. 5,801,005
U.S. Pat. No. 5,824,311
U.S. Pat. No. 5,830,880
U.S. Pat. No. 5,846,945
Urayama et al., Rapid Commun. Mass Spectrom., 24:613-620, 2010.
van den Berg R et al., BMC Genomics 7:142, 2006.
van Heerden et al., Mayo Clin. Proc., 56:601-606, 1981.
Veselkov et al., Anal Chem 83:5864-72, 2011.
Weljie A M, Jirik F R, J Biochem Cell Biol 43:981-9, 2011.
Weljie et al., Anal. Chem., 78:4430-4442, 2006.
Wen et al., J. Hepatol., 52(2):228-233, 2009.
Wishart et al., Nucleic Acids Res., 37:D603-610, 2009.
Wishart, Methods Molec. Biol., 593:283-313, 2010.
Xia and Wishart, Nat Prot 6:743-60, 2011.
Xia and Wishart, Nucleic Acids Res 38:W71-W77, 2010.
Xue et al., Clin. Chem. Lab. Med., 47(3):305-310. 2009.
Yeo et al., Ann Surg 226:248-57, 1997.

What is claimed:

1. A method of distinguishing pancreatic cancer and periampullary adenocarcinoma from benign pancreatic lesions in a subject comprising:
(a) obtaining a blood, plasma or serum sample;
(b) determining the levels of 4 or more of the biomarkers set forth in Table 3 in said sample; and
(c) assigning to said sample a classification as
(i) pancreatic cancer or periampullary adenocarcinoma where one or more markers indicating pancreatic cancer or periampullary adenocarcinoma are elevated; and
(ii) benign pancreatic lesion where one or more markers indicating benign pancreatic lesions are elevated.

2. The method of claim 1, wherein step (b) comprises determining the levels of Galactose, Unmatched RI:1007.82 QI: 67/82/83, Isopropanol, Mannose, Trimethylamine-N-oxide, Arabitol, Threitol, Succinate, Trehalose-alpha, Match RI:2018.25 QI: 191/217/305/318/507, Tridecanol, Azelaic acid, Unmatched RI:2475.33 QI: 73/375/376, Pyroglutamate, Isoleucine, Tyrosine, Arginine, Unmatched RI:1913.88 QI: 156/174/317, Alanine, Creatine, Lysine, Unmatched RI:1971.99 QI: 185/247/275; and step (c) comprises assigning to said sample a classification as pancreatic cancer or periampullary adenocarcinoma where a pluraliity of Galactose, Unmatched RI:1007.82 QI: 67/82/83, Isopropanol, Mannose, Trimethylamine-N-oxide, Arabitol, Threitol, Succinate, and Trehalose-alpha levels are elevated; and benign pancreatic lesion where a plurality of Match RI:2018.25 QI: 191/217/305/318/507, Tridecanol, Azelaic acid, Unmatched RI:2475.33 QI: 73/375/376, Pyroglutamate, Isoleucine, Tyrosine, Arginine, Unmatched RI:1913.88 QI: 156/174/317, Alanine, Creatine, Lysine, Unmatched RI:1971.99 QI: 185/247/275 levels are elevated.

3. The method of claim 1, wherein the information was determined using gas chromatography/mass spectrometry.

4. The method of claim 1, wherein the information was determined using nuclear magnetic resonance.

5. The method of claim 1, wherein the information was determined using nuclear magnetic resonance and gas/chromatography/mass spectrometry.

6. The method of claim 1, wherein information on the level of at least 10 of the metabolites from Table 3 is determined.

7. The method of claim 1, wherein information on the level of at least 15 of the metabolites from Table 3 is determined.

8. The method of claim 1, wherein information on the level of at least 20 of the metabolites from Table 3 is determined.

9. The method of claim 1, wherein information on the level of at least 25 of the metabolites from Table 3 is determined.

10. The method of claim 1, wherein information on the level of all of the metabolites from Table 3 is determined.

11. The method of claim 1, wherein said levels are compared to a pre-determined standard.

12. The method of claim 1, wherein said levels are determined from a serum, plasma or blood sample from a non-cancer subject.

13. The method of claim 1, further comprising obtaining said serum, plasma or blood sample from said subject.

14. The method of claim 1, wherein said pancreatic cancer is metastatic.

15. The method of claim 1, wherein said pancreatic cancer is localized.

16. The method of claim 1, further comprising treating said subject for pancreatic cancer.

17. The method of claim 16, wherein said treatment is surgery, chemotherapy or both.

18. The method of claim 1, wherein the non-pancreatic cancer is a benign pancreatic lesion.

19. The method of claim 1, wherein the non-pancreatic cancer is a periampullary adenocarcinoma.

20. The method of claim 1, where the periampullary adenocarcinoma is ampullary cancer, bile duct cancer or duodenal cancer.
